SEQUENCE LISTING (38 sequences)

SEQ ID NO: 1 - DNA, 1584 bp, synthetic construct
  source 1..1584: mol_type = other DNA; note = Gene encoding pck integrated into the yeeL locus
atgaactcagttgatttgaccgctgatttacaagccttattaacatgtccaaatgtgcgtcataatttatcagcagcacagctaacagaaaaagtcctctcccgaaacgaaggcattttaacatccacaggtgctgttcgcgcgacaacaggcgcttacacaggacgctcacctaaagataaattcatcgtggaggaagaaagcacgaaaaataagatcgattggggcccggtgaatcagccgatttcagaagaagcgtttgagcggctgtacacgaaagttgtcagctatttaaaggagcgagatgaactgtttgttttcgaaggatttgccggagcagacgagaaatacaggctgccgatcactgtcgtaaatgagttcgcatggcacaatttatttgcgcggcagctgtttatccgtccggaaagaaatgataagaaaacagttgagcagccgttcaccattctttctgctccgcatttcaaagcggatccaaaaacagacggcactcattccgaaacgtttattattgtctctttcgaaaagcggacaattttaatcggcggaactgagtatgccggtgaaatgaagaagtccattttctccattatgaatttcctgctgcctgaaagagatattttatctatgcactgctccgccaatgtcggtgaaaaaggcgatgtcgcccttttcttcggactgtcaggaacaggaaagaccaccctgtcggcagatgctgaccgcaagctgatcggtgacgatgaacatggctggtctgatacaggcgtctttaatattgaaggcggatgctacgctaagtgtattcatttaagcgaggaaaaggagccgcaaatctttaacgcgatccgcttcgggtctgttctcgaaaatgtcgttgtggatgaagatacacgcgaagccaattatgatgattccttctatactgaaaacacgcgggcagcttacccgattcatatgattaataacatcgtgactccaagcatggccggccatccgtcagccattgtatttttgacggctgatgccttcggagtcctgccgccgatcagcaaactaacgaaggagcaggcgatgtaccattttttgagcggttacacgagtaagcttgccggaaccgaacgtggtgtcacgtctcctgaaacgacgttttctacatgcttcggctcaccgttcctgccgcttcctgctcacgtctatgctgaaatgctcggcaaaaagatcgatgaacacggcgcagacgttttcttagtcaataccggatggaccgggggcggctacggcacaggcgaacgaatgaagctttcttacactagagcaatggtcaaagcagcgattgaaggcaaattagaggatgctgaaatgataactgacgatattttcggcctgcacattccggcccatgttcctggcgttcctgatcatatccttcagcctgaaaacacgtggaccaacaaggaagaatacaaagaaaaagcagtctaccttgcaaatgaattcaaagagaactttaaaaagttcgcacataccgatgccatcgcccaggcaggcggccctctcgtataa

SEQ ID NO: 2 - DNA, 28 bp, synthetic construct
  source 1..28: mol_type = other DNA; note = DNA sequence used for RBS optimization of pck
catcagataggtgtaaggaggtttagat

SEQ ID NO: 3 - DNA, 3444 bp, synthetic construct
  source 1..3444: mol_type = other DNA; note = Gene encoding pyc integrated into the yjhE locus
atgtcacaacagtctattcaaaaagttcttgttgcaaacagaggggaaatcgctatccgcgtatttcgggcctgcacagaactgaatatccgtacggtagcgatctattctaaagaagacagcggatcttaccacagatacaaagccgatgaagcatacctggtcggcgaagggaaaaagccgattgacgcttatcttgatattgaaggcatcatcgagattgcaaaacgcaaccatgtggatgccatccatccaggctacggcttcctgtcggaaaacattcagtttgctaagcggtgtgaagaggaaggcatcatctttatcggaccgacctccgagcacctcgatatgtttggagacaaagtaaaagcccgcgaacaagctgaaaaagctggaattccggtcataccggggagcgacggaccagtggcggatatagcggaagtgaaacaatttgcggaaaagttcggatatccgtttatcattaaagcgtcgcttggcggcggcgggcgcggcatgcggatcgtcagggacgaatcggagctggtggagtcctataatagggcgaaatcagaggcgaaagcggcctttggcaatgatgaagtttatgtcgaaaagctgattgaaaagccgaagcacattgaagttcaagtcatcggagataaagaagggaacgtggttcacctttacgaccgcgactgctctgtgcaaaggcgtcatcaaaaggtcatcgaagtgaagccgagcgtttcgctttctgaatccctccgggaaaagatttgcgatgctgccgttaagcttgcgaagaatgttgaatatgtcaatgccggtacagtcgaatttttagttgcgaacgatgagtttttctttattgaagtgaacccgcgtgttcaggtggagcataccattacggaaatggtaacgggcgtcgatatcgttcagacgcaaatcctgatcgctgccggtctcagtctggacagcagcgaaatcagcattccgaaccaggatgcgatcacgctgcacggatatgcgatccagtcaagggttacgactgaagatccgtcaaacaatttcatgcctgacacaggcaaaatcatggcatatcgctcaggcggcggtttcggggtgaggcttgataccggaaacagttttcagggcgctgtcattacgccttattacgattcgctgcttgttaagctttcaacttgggcgctgacgtttgaacaggcggcagccaaaatggtccgcaaccttcaggaattcaggatcaggggaatcaaaacgaacattcctttccttgaaaatgtggcgaaacacgaaaagtttcttacagggcaatacgacacgtcttttatcgatacaacgccagaactttttgtctttcctaagcagagagaccgcggaacgaaaatgctgacatatatcgggaacgtaacggtcaacggcttcccgggcatcgataaaaagaagaagcccgagtttgataagccgcagatcgtcaaaacagatgtcgatcagccaatcgcaagcggaacaaaacagattcttgatgaacgcggagccgaagggctcgtcaaatgggtgaaagatcaagaggaagtgctcctcactgatacgacgttccgcgacgcccatcagtcattgcttgcgacaagagtcagaacgcatgacctgaaaaaaatcgccaatccgacggctgcgctctggccagagcttttcagtcttgaaatgtggggcggcgccacatttgatgtcgcttaccgtttcttgaaagaagatccgtggaaaaggctcgaggagctgcggaaggaaattccgaatacgatgtttcaaatgcttttgagatcttcgaatgccgtcgggtatacaaactaccctgacaacctgattaaaaagttcgtcagcgaatcggctgcggccggaatcgatgtattccgcatttttgacagcttgaactgggttaaagggatgacgctcgccattgatgcagtgcgcgagtcaggcaagcttgccgaagcggcgatctgctatacaggggacattctcgatccgaacagaagcaagtataatcttgaatattatacgtcaatggcaaaagagcttgaagctgcgggggcgcatattctcggcattaaagatatggccggcctgctgagacctcaagcggcatacgaactggtgtcggctttgaaagagacgatcgacattccgatccacttgcatacacacgacacgagcggcaacggtatttttatgtatgcgaaggcgatagaagcaggcgtcgacatcgtcgacgtagcggtcagctcgatggcgggtctgacatcacagccaagcgcaagctcgctttaccatgcgcttgaaggagataaacgccgtccgcagttcaatgtcgatgcggtagagtcgttgtctcaatattgggagtctgtcagaaaatattacagcgagtttgagagcggcatgattgcgcctcataccgaaatttacaagcatgaaatgcctggcggccaatacagcaaccttcagcagcaggctaagggagtcggcctcggcgaccgctggaatgaagtgaaagaaatgtacagccgggtcaaccacctgttcggggacatcgtaaaggttacgccgtcatctaaagtcgtcggggatatggcgctttacatggtgcaaaataacctgacggaagatgatatttacgaaaggggagaatctctcgattttcctgactcggttgtcgagctttttaaagggtatatcggtcagcctcacggcggattccctgagaaattgcaaaagctgattttaaaagggcaggagccgattacagtacgccctggagaactgcttgaaccggtttcatttgacgctataaaagcggaattcttggaaaagcatggtatggagctttccgaccaggatgctgttgcatatgcgctttatccgaaagtgtttaccgagtatgtcaagacagcggaactgtacggggatatctctgtgcttgatacaccaaccttcctctacggcatgaccctcggcgaagaaatcgaggtcgagattgaaagagggaaaacgctgatcgtcaaacttgtctctatcggggaaccgcgtccggatgcgacgagagtcgtttattttgaactgaacggccagcctcgcgaagtggtcattaaagacgaaagcattaaatcgtccgttcatcaaaaggtgaaagccgaccgttcaaacccgaatcatatcgcggcatctatgccgggaacggtaatcaagcttcttgtaagcaaaggggaccaagtgaagaagggcgatcatttgatgatcaatgaagcgatgaaaatggaaacgaccgttcaggctccgttttcaggaactgttgaaaacattcacgttacaaacggagaagccattcaaaccggcgaccttctcattgaactgaaaaaataa

SEQ ID NO: 4 - DNA, 28 bp, synthetic construct
  source 1..28: mol_type = other DNA; note = DNA sequence used for RBS optimization of pyc
caacagataggtgtaaggaggttgagat

SEQ ID NO: 5 - DNA, 18 bp, synthetic construct
  source 1..18: mol_type = other DNA; note = Nucleotide sequence of the gene encoding thrABA144DC
tttcacacaggaaacaga

SEQ ID NO: 6 - DNA, 29 bp, synthetic construct
  source 1..29: mol_type = other DNA; note = DNA sequence used for RBS optimization of thrABA144DC
cggtaaagatatcgataaggaggtttttt

SEQ ID NO: 7 - DNA, 20 bp, synthetic construct
  source 1..20: mol_type = other DNA; note = Primer yeeL-U-F
aagaaatccgacgccaaagg

SEQ ID NO: 8 - DNA, 70 bp, synthetic construct
  source 1..70: mol_type = other DNA; note = Primer yeeL-U-R
atccgctcacaattccacacattatacgagccggatgattaattgtcaacctaacctcgcctccctactg

SEQ ID NO: 9 - DNA, 42 bp, synthetic construct
  source 1..42: mol_type = other DNA; note = Primer yeeL-D-F
caggcggccctctcgtataatggcaagtgcctataatacccc

SEQ ID NO: 10 - DNA, 22 bp, synthetic construct
  source 1..22: mol_type = other DNA; note = Primer yeeL-D-R
tcatctagtcccgcaaactcaa

SEQ ID NO: 11 - DNA, 83 bp, synthetic construct
  source 1..83: mol_type = other DNA; note = Primer pck-1
ctcgtataatgtgtggaattgtgagcggataacaacatcagataggtgtaaggaggtttagatatgaactcagttgatttgac

SEQ ID NO: 12 - DNA, 22 bp, synthetic construct
  source 1..22: mol_type = other DNA; note = Primer pck-2
cttatcatttctttccggacgg

SEQ ID NO: 13 - DNA, 22 bp, synthetic construct
  source 1..22: mol_type = other DNA; note = Primer pck-3
ccgtccggaaagaaatgataag

SEQ ID NO: 14 - DNA, 20 bp, synthetic construct
  source 1..20: mol_type = other DNA; note = Primer pck-4
ttatacgagagggccgcctg

SEQ ID NO: 15 - DNA, 23 bp, synthetic construct
  source 1..23: mol_type = other DNA; note = Primer yjhE-U-F
ccagtttaataagaaaggagacg

SEQ ID NO: 16 - DNA, 74 bp, synthetic construct
  source 1..74: mol_type = other DNA; note = Primer yjhE-U-R
ttgttatccgctcacaattccacacattatacgagccggatgattaattgtcaatgtcgtgaactgtgagacga

SEQ ID NO: 17 - DNA, 53 bp, synthetic construct
  source 1..53: mol_type = other DNA; note = Primer yjhE-D-F
ttgaactgaaaaaataaaagtcgaatcagggctgaagtggcacactgaatttg

SEQ ID NO: 18 - DNA, 22 bp, synthetic construct
  source 1..22: mol_type = other DNA; note = Primer yjhE -D-R
acaacagaccgagaaagacact

SEQ ID NO: 19 - DNA, 67 bp, synthetic construct
  source 1..67: mol_type = other DNA; note = Primer pyc-1
aattgtgagcggataacaacaacagataggtgtaaggaggttgagatatgtcacaacagtctattca

SEQ ID NO: 20 - DNA, 21 bp, synthetic construct
  source 1..21: mol_type = other DNA; note = Primer pyc-2
aacgctcggcttcacttcgat

SEQ ID NO: 21 - DNA, 21 bp, synthetic construct
  source 1..21: mol_type = other DNA; note = Primer pyc-3
atcgaagtgaagccgagcgtt

SEQ ID NO: 22 - DNA, 17 bp, synthetic construct
  source 1..17: mol_type = other DNA; note = Primer pyc-4
ttattttttcagttcaa

SEQ ID NO: 23 - DNA, 22 bp, synthetic construct
  source 1..22: mol_type = other DNA; note = Primer ydeu-U-F
cataagcgggaagggtatcgtg

SEQ ID NO: 24 - DNA, 75 bp, synthetic construct
  source 1..75: mol_type = other DNA; note = Primer ydeu-U-R
ttgttatccgctcacaattccacacattatacgagccggatgattaattgtcaattgaaccgtgccgccattctc

SEQ ID NO: 25 - DNA, 51 bp, synthetic construct
  source 1..51: mol_type = other DNA; note = Primer ydeu-D-F
tgatgatgaatcatcagtaaaccgtataagccgcatgtcgagatggcatgc

SEQ ID NO: 26 - DNA, 22 bp, synthetic construct
  source 1..22: mol_type = other DNA; note = Primer ydeu -D-R
atgtcgtgagcgtggtattgtc

SEQ ID NO: 27 - DNA, 69 bp, synthetic construct
  source 1..69: mol_type = other DNA; note = Primer thrA-F
gaattgtgagcggataacaacggtaaagatatcgataaggaggttttttatgcgagtgttgaagttcgg

SEQ ID NO: 28 - DNA, 20 bp, synthetic construct
  source 1..20: mol_type = other DNA; note = Primer thrB-R
aaacacgggtccacgttgtc

SEQ ID NO: 29 - DNA, 20 bp, synthetic construct
  source 1..20: mol_type = other DNA; note = Primer thrB-F
gacaacgtggacccgtgttt

SEQ ID NO: 30 - DNA, 20 bp, synthetic construct
  source 1..20: mol_type = other DNA; note = Primer thrC -R
ttactgatgattcatcatca

SEQ ID NO: 31 - DNA, 27 bp, synthetic construct
  source 1..27: mol_type = other DNA; note = Primer PGRB-F
gttttagagctagaaatagcaagttaa

SEQ ID NO: 32 - DNA, 19 bp, synthetic construct
  source 1..19: mol_type = other DNA; note = Primer PGRB-R
attatacctaggactgagc

SEQ ID NO: 33 - DNA, 56 bp, synthetic construct
  source 1..56: mol_type = other DNA; note = Primer sgRNA-yeel-1
agtcctaggtataatactagtaacacagcaatacggtacgcgttttagagctagaa

SEQ ID NO: 34 - DNA, 56 bp, synthetic construct
  source 1..56: mol_type = other DNA; note = Primer sgRNA-yeel-2
ttctagctctaaaacgcgtaccgtattgctgtgttactagtattatacctaggact

SEQ ID NO: 35 - DNA, 56 bp, synthetic construct
  source 1..56: mol_type = other DNA; note = Primer sgRNA-ydeu-1
agtcctaggtataatactagttatctgaccagtaaatgggagttttagagctagaa

SEQ ID NO: 36 - DNA, 56 bp, synthetic construct
  source 1..56: mol_type = other DNA; note = Primer sgRNA-ydeu-2
ttctagctctaaaactcccatttactggtcagataactagtattatacctaggact

SEQ ID NO: 37 - DNA, 56 bp, synthetic construct
  source 1..56: mol_type = other DNA; note = Primer sgRNA-yjhE-1
agtcctaggtataatactagtgcctatccgggctgtcccgagttttagagctagaa

SEQ ID NO: 38 - DNA, 56 bp, synthetic construct
  source 1..56: mol_type = other DNA; note = Primer sgRNA-yjhE-2
ttctagctctaaaactcgggacagcccggataggcactagtattatacctaggact

(19) United States
(12) Patent Application Publication
(10) Pub. No.: US 2024/0018557 A1
(43) Pub. Date: Jan. 18, 2024

(54) RECOMBINANT NUCLEIC ACID OF ESCHERICHIA COLI, RECOMBINANT ESCHERICHIA COLI AND CULTURING METHOD THEREOF, AND METHOD FOR BIOSYNTHESIZING L-THREONINE THEREBY

(71) Applicant: Jiangnan University, Wuxi City (CN)

(72) Inventors: Zhiming RAO, Wuxi City (CN); Zhenqiang ZHAO, Wuxi City (CN); Meijuan XU, Wuxi City (CN); Xian ZHANG, Wuxi City (CN); Taowei YANG, Wuxi City (CN)

(73) Assignee: Jiangnan University, Wuxi City (CN)

(21) Appl. No.: 17/930,223

(22) Filed: Sep. 7, 2022

(30) Foreign Application Priority Data
May 19, 2022 (CN) 202210552528.4

(51) Int. Cl. C12P 13/08; C12N 15/70

(52) U.S. Cl. CPC C12P 13/08; C12N 15/70; C12Y 604/01001; C12Y 401/01032; C12N 2800/22

(57) ABSTRACT

The present disclosure provides a recombinant nucleic acid of Escherichia coli, a recombinant E. coli, a culturing method thereof, and a method for biosynthesizing L-threonine thereby, and relates to the technical field of bioengineering. The recombinant nucleic acid of E. coli of the present disclosure, including the gene encoding phosphoenolpyruvate carboxykinase (pck), the gene encoding pyruvate carboxylase (pyc) and the gene encoding threonine operon, is transformed into E. coli to obtain a recombinant E. coli LMT4 strain that takes glucose as a substrate. Using the LMT4 for fermentative production may significantly improve the L-threonine yield and glucose conversion rate, laying a foundation for the industrial production of L-threonine.

### CROSS REFERENCE TO RELATED APPLICATION

This patent application claims the benefit and priority of Chinese Patent Application No. 202210552528.4, entitled “Recombinant nucleic acid of Escherichia coli, recombinant Escherichia coli and culturing method thereof, and method for biosynthesizing L-threonine thereby” filed on May 19, 2022, the disclosure of which is incorporated by reference herein in its entirety as part of the present application.

### REFERENCE TO AN ELECTRONIC SEQUENCE LISTING

The contents of the electronic sequence listing (GWP2021091002.xml; Size: 49,152 bytes; and Date of Creation: Sep. 2, 2022) is herein incorporated by reference in its entirety.

### TECHNICAL FIELD

The present disclosure belongs to the technical field of bioengineering, and in particular relates to a recombinant nucleic acid of Escherichia coli, a recombinant Escherichia coli and a culturing method thereof, and a method for biosynthesizing L-threonine thereby.

### BACKGROUND ART

Threonine is an essential amino acid, which is mainly used in medicine, chemical reagents, food fortifiers, feed additives and so on. Because the structure of threonine contains hydroxyl groups, it has a water-holding effect on human skin, can bind with oligosaccharide chains to play an important role in protecting cell membranes, and can promote phospholipid synthesis and fatty acid oxidation in vivo. Threonine formulation has the medicinal effect of promoting human development and anti-fatty liver, and is a component of compound amino acid infusion. Meanwhile, threonine is the second limiting amino acid in pig feed and the third limiting amino acid in poultry feed. In recent years, the global population's demand for meat products has shown a trend of continuous and rapid growth, so the market demand for L-threonine is also increasing.

At present, the methods for producing L-threonine mainly include chemical synthesis method, proteolysis method and microbial fermentation method, where the microbial fermentation method with low production cost, high production intensity and little environmental pollution has become the most widely used method in industrial production of L-threonine. The strains currently producing L-threonine mainly include Escherichia coli, Corynebacterium glutamicum, and Serratia marcescens. Sang Yup Lee et al. started from E. coli W3100 (lacI-) by using the method of systems biology, and the constructed engineering strain produced 82.4 g/L of L-threonine after 50 h of fermentation with a glucose conversion rate of 39.3%. Jianjun Qiao of Tianjin University used THRD as the starting strain to improve the L-threonine yield through two-stage carbon allocation and cofactor generation strategy. The obtained strain produced 70.8 g/L of L-threonine in 40 h with a glucose conversion rate of 40.4%. Qiong Shen from East China University of Science and Technology constructed genetically engineered bacteria E. coli VNBKB.3507 by strengthening the key enzymes of L-threonine synthesis pathway and the genes related to L-threonine secretion, which produced 82.4 g/L of L-threonine after 48 h of fermentation. Xiaoyuan Wang of Jiangnan University modified E. coli TWF001 through metabolic engineering. After 36 h of shake flask culture, the obtained modified strain could produce 15.85 g/L L-threonine with a glucose conversion rate of 53%.

At present, the E. coli strains used to produce threonine there still has much room for improvement in the L-threonine yield and the glucose conversion rate, and the method of overexpressing key genes through plasmids requires adding antibiotics during the fermentation process to maintain the stability of the plasmid, which not only increases the potential safety hazard but also increases the fermentation cost. Therefore, the development of a plasmid-free recombinant strain for synthesizing L-threonine with high efficiency is of great significance to industrial production.

### SUMMARY

In view of this, the objective of the present disclosure is to provide a recombinant nucleic acid of E. coli, recombinant E. coli and culture method thereof, and method for biosynthesizing L-threonine, based on systematic metabolic engineering modification and optimization strategy to obtain a recombinant strain of E. coli with high synthesis efficiency of L-threonine acid.

In order to achieve the above objective of the present disclosure, provides the following technical solutions:

The present disclosure provides a recombinant nucleic acid of E. coli, comprising a gene encoding phosphoenolpyruvate carboxykinase (pck), a gene encoding pyruvate carboxylase (pyc) and a gene encoding threonine operon.

In some embodiments, expression of the gene encoding pck, the gene encoding pyc and the gene encoding threonine operon is initiated by Trc promoter.

In some embodiments, the gene encoding pck is derived from Bacillus subtilis;

The gene encoding pyc is derived from Bacillus licheniformis.

In some embodiments, the pck is ribosome binding site (RBS) optimized and glycine is mutated to arginine at position 143;

The pyc is RBS optimized and alanine is mutated to lysine at position 247.

In some embodiments, the threonine operon is RBS optimized and alanine is mutated to aspartic acid at position 144.

The present disclosure also provides a recombinant E. coli comprising the above recombinant nucleic acid, in which the recombinant E. coli overexpresses pck, pyc and threonine operon.

In some embodiments, the basic strain of the recombinant E. coli includes E. coli K-12 W3110.

The present disclosure also provides a culturing method for the above recombinant E. coli, including the following steps: inoculating the recombinant E. coli on a seed medium for culturing to obtain a seed liquid; the seed medium includes components with the following concentrations: dried corn steep liquor powder 5 g/L, glucose 20 g/L, yeast powder 5 g/L, KH₂PO₄ 2 g/L, magnesium sulfate 1 g/L, FeSO₄·7H₂O 20 mg/L and MnSO₄·H₂O 20 mg/L.

The present disclosure also provides a use of the above recombinant E. coli in the biosynthesizing of L-threonine, in which the recombinant E. coli takes glucose as a fermentation substrate.

The present disclosure also provides a method for biosynthesizing L-threonine, including the following steps: inoculating the seed liquid obtained by the above culturing method in a fermentation medium, performing an aerobic fermentation, in which an obtained L-threonine is contained in fermentation liquid;

the fermentation medium includes the components of the following concentrations: glucose 20 g/L, potassium dihydrogen phosphate 2 g/L, yeast powder 3 g/L, betaine 1 g/L, magnesium sulfate 1 g/L, FeSO₄·7H₂O 10 mg/L, MnSO₄—H₂O 10 mg/L, dried corn steep liquor powder 8 g/L and vitamin B1 10 mg/L.

Beneficial effects: The present disclosure provides a recombinant nucleic acid of E. coli, including the gene encoding pck, the gene encoding pyc and the gene encoding threonine operon. The genome of E. coli is modified by using the recombinant nucleic acid, and the plasmid is genetically modified by CRISPR-Cas9 technology. Therefore, a plasmid-free recombinant E. coli LMT4 is obtained for the final fermentation process to produce threonine. Using the recombinant E. coli LMT4 for fermentation and production, with glucose as a substrate, significantly improves the L-threonine yield and the glucose conversion rate, laying a foundation for the industrial production of L-threonine. In the example of the present disclosure, with the recombinant E. coli LMT4 for fermentation in a 5 L fermentor for 48 h and glucose as a substrate, 160 g/L threonine was produced with a glucose conversion rate of 60%, indicating that the recombinant strain for synthesizing L-threonine with high efficiency of the present disclosure may have a wide range of industrial application prospects.

### BRIEF DESCRIPTION OF THE DRAWINGS

FIG. 1 is a graph showing the L-threonine production of the recombinant strain LMT4 in 5 L fermenter.

FIG. 2 is a growth curve diagram of recombinant strain LMT4 in a 5 L fermenter.

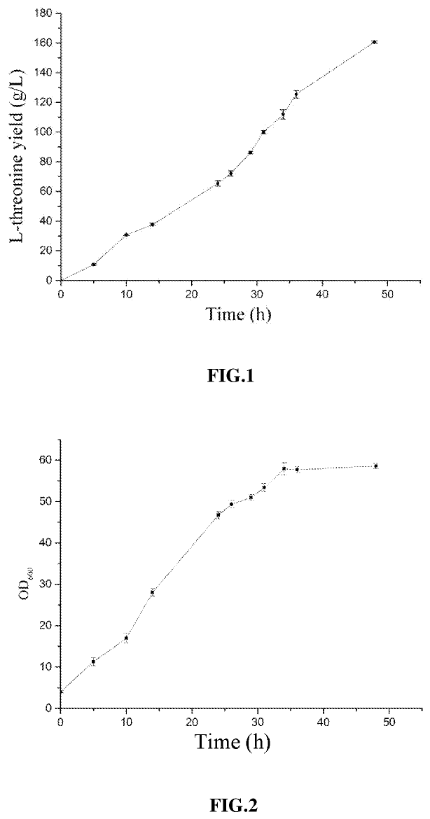

### DETAILED DESCRIPTION OF THE EMBODIMENTS

The present disclosure provides a recombinant nucleic acid of E. coli, including a gene encoding pck, a gene encoding pyc and a gene encoding threonine operon.

In some embodiments of the present disclosure, E. coli is used as the starting strain, and by means of gene editing, the pseudogene yeeL of the starting strain is knocked out and the gene encoding pck is integrated into the yeeL locus; the pseudogene yjhE of the starting strain is knocked out and the gene encoding pyc is integrated into the yjhE locus; and the pseudogene ydeu of the starting strain is knocked out and the gene encoding threonine operon is integrated into the ydeu locus. In some embodiments of the present disclosure, expressions of the gene encoding pck, the gene encoding pyc and the gene encoding threonine operon is initiated by Trc promoter. The method of gene editing is not particularly limited in the present disclosure, and in some embodiments it includes CRISPR Cas9.

In some embodiments of the present disclosure, the gene encoding pck is derived from B. subtilis. In some embodiments, before being integrated into the yeeL locus, the gene encoding pck is subjected to RBS optimization and mutation from glycine to arginine at position 143. In some embodiments, the RBS optimization of the present disclosure is to replace the RBS sequence located in the upstream of the gene encoding pck for regulating pck, with SEQ ID NO: 2: CATCAGATAGGTGTAAGGAGGTTTAGAT. In some embodiments, after the RBS optimization and the mutation of the present disclosure, the complete sequence of the gene encoding pck integrated into the yeeL locus is set forth in SEQ ID NO:1.

In some embodiments, the gene encoding pyc of the present disclosure is derived from B. licheniformis. In some embodiments, before being integrated into the yjhE locus, the gene encoding pyc is also subjected to RBS optimization and mutation from alanine to lysine at position 247. In some embodiments, the RBS optimization is to replace the RBS sequence located in the upstream of the gene encoding pyc for regulating pyc, and in some other embodiments, the nucleotide sequence of the replaced RBS sequence is set forth in SEQ ID NO: 4: CAACAGATAGGTGTAAGGAGGTTGAGAT. After the RBS optimization and mutation of the present disclosure, in some embodiments, the complete sequence of the gene encoding pyc integrated into the yjhE locus is set forth in SEQ ID NO:3.

The alanine is mutated to aspartic acid at the position 144 of the threonine operon of the present disclosure (thrABA144DC). In some embodiments, after the mutation, the nucleotide sequence of the gene encoding thrABA144DC is set forth in SEQ ID NO:5: TTTCACACAGGAAACAGA; meanwhile, RBS optimization is performed on the sequence set forth in SEQ ID NO:5, and in some embodiments, the RBS optimization is performed by replacing the RBS sequence set forth in SEQ ID NO:5 with the sequence set forth in SEQ ID NO:6: CGGTAAAGATATCGATAAGGAGGTTTTTT, and then the mutated and RBS-optimized thrABA144DC is integrated into the ydeu locus.

The present disclosure further provides a recombinant E. coli including the above recombinant nucleic acid, and the recombinant E. coli overexpresses pck, pyc and threonine operon.

In some embodiments, the basic strain of the recombinant E. coli of the present disclosure includes E. coli K-12 W3110, and the basic strain lacks the DNA-binding transcription inhibitor LacI, the threonine leader peptide encoding gene thrL, and the Na(+)/serine-threonine symporter gene sstT, threonine dehydrogenase tdh, threonine transporter tdcC. In some embodiments, the E. coli W3110 of the present disclosure is purchased from Beina Bio.

The present disclosure also provides a method for constructing the recombinant E. coli. In some embodiments, the method of CRISPR Cas9 is used for construction (FIG. 4), and in some other embodiments, the method includes the following steps. (1) Upstream and downstream homologous arms of the pseudogene yeeL from the genome of Escherichia coli K-12 W3110 are amplified by PCR; (2) primers pck-1, pck-2, pck-3 and pck-4 in Table 1 are used to amplify the pck^{G143R} gene from the Bacillus subtilis genome to obtain fragments 1-PCK and 2-PCK, and then with pck-1 and the pck-4 as primers and the fragment 1-pck^{G143R} and 2-pck^{G143R} as templates, the fragments 1-pck^{G143R} and 2-pck^{G143R} are fused into pck^{G143R}, where the pck-1 primer contains the optimized RBS sequence, the pck-2 and the pck-3 are gene point mutation primers of pck^{G143R}, and the RBS optimization and point mutation of pck^{G143R} gene are conducted by PCR; (3) the upstream and downstream homology arms of yeeL, and the pck^{G143R} fragment initiated by the Trc promoter are fused to obtain an U-pck^{G143R}_D fragment; (4) the obtained fusion fragment U-pck^{G143R}_D and the vector containing yeel-sgRNA are transformed into the recombinant E. coli K-12 W3110 (LMT1 for short) to obtain a recombinant strain, in which the pseudogene yeeL is knocked out and the B. subtilis-derived pck^{G143R} initiated by the Trc promoter is integrated into the yeeL locus, and the yeel-sgRNA vector is removed to obtain a recombinant strain LMT2;

(5) upstream and downstream homology arms of the pseudogene yjhE from the genome of E. coli K-12 W3110 are amplified by PCR; (6) primers pyc-1, pyc-2, pyc-3 and pyc-4 in Table 1 are used to amplify the pyc^{A247K} gene from the B. licheniformis genome to obtain the fragments 1-pyc^{A247K} and 2-pyc^{A247K}, and then with the pyc-1 and the pyc-4 as primers, and the fragments 1-pyc^{A247K} and 2-pyc^{A247K} as templates, the 1-pyc^{A247K} and 2-pyc^{A247K} are fused into pyc^{A247K}, where the pyc-1 primer contains the optimized RBS sequence, the pyc-2 and the pyc-3 are gene point mutation primers of pyc^{A247K}, and the RBS optimization and point mutation of pyc^{A247K} gene RBS are completed by PCR; (7) the upstream and the downstream homology arms of yjhE, and the pyc^{A247K} fragment of initiated by the Trc promoter are fused to obtain an U-pyc^{A247K}-D fragment; (8) the obtained fusion fragment U-pyc^{A247K}-D and the vector containing yjhE-sgRNA are transformed into the recombinant strain LMT2 to obtain a recombinant strain, in which the pseudogene yjhE is knocked out and the pycA247K initiated by the Trc promoter is integrated into the yjhE locus, and the yjhE-sgRNA vector is removed to obtain a recombinant strain LMT3;

(9) upstream and downstream homology arms of the pseudogene ydeu from the genome of E. coli K-12 W3110 are amplified by PCR; (10) primers thrA-F, thrB-R and thrB-F, thrC-R in Table 1 are used to amplify the thrAB^{A144D}C gene cluster of the threonine operon initiated by the Trc promoter by PCR from the genome of E. coli K-12 W3110 to obtain the fragments 1-thrAB^{A144D}C and 2-thrAB^{A144D}C, and then with thrA-F and the thrC-R as primers, and the fragments 1-thrAB^{A144D}C and 2-thrAB^{A144D}C as templates, the fragments 1-thrAB^{A144D}C and 2_thrAB^{A144D}C are fused into thrAB^{A144D}C, where the thrA-F primer contains the optimized RBS sequence, the thrB-R and the thrB-F are gene point mutation primers of the thrAB^{A144D}C, and the RBS optimization and point mutation of the threonine operon thrAB^{A144D}C gene are conducted by PCR; (11) the upstream and downstream homology arms of ydeu and the thrAB^{A144D}C gene cluster fragment of the threonine operon initiated by the Trc promoter are fused to obtain an U-thrAB^{A144D}C-D fragment; (12) the obtained fusion fragment U-thrAB^{A144D}C-D and the vector containing ydeu-sgRNA are transformed into the recombinant strain LMT3 to obtain a recombinant strain, in which the pseudogene ydeu is knocked out and the threonine operon thrAB^{A144D}C gene cluster initiated by the Trc promoter is integrated into the ydeu locus, and the ydeu-sgRNA vector is removed to obtain a recombinant E. coli strain LMT4.

In the present disclosure, in order to complete the construction of the recombinant E. coli, the primers shown in Table 1 are used.

TABLE 1
Primers used

| | | SEQ ID |
|---|---|---|
| Primer | Sequence from 5′ to 3′ | NO |
| yeeL-U-F | AAGAAATCCGACGCCAAAGG | 7 |
| yeeL-U-R | ATCCGCTCACAATTCCACACATTATACGAGCCGGATGAT | 8 |
| | TAATTGTCAACCTAACCTCGCCTCCCTACTG | |
| yeeL-D-F | caggcggccctctcgtataaTGGCAAGTGCCTATAATACCCC | 9 |
| yeeL-D-R | TCATCTAGTCCCGCAAACTCAA | 10 |
| pck-1 | CTCGTATAATGTGTGGAATTGTGAGCGGATAACAACAT | 11 |
| | CAGATAGGTGTAAGGAGGTTTAGATatgaactcagttgatttgac | |
| pck-2 | cttatcatttcTttccggacgg | 12 |
| pck-3 | ccgtccggaaAgaaatgataag | 13 |
| pck-4 | ttatacgagagggccgcctg | 14 |
| yjhE-U-F | CCAGTTTAATAAGAAAGGAGACG | 15 |
| yjhE-U-R | TTGTTATCCGCTCACAATTCCACACATTATACGAGCCGG | 16 |
| | ATGATTAATTGTCAATGTCGTGAACTGTGAGACGA | |
| yjhE-D-F | ttgaactgaaaaaataaAAGTCGAATCAGGGCTGAAGTGGCACA | 17 |
| | CTGAATTTG | |
| yjhE-D-R | ACAACAGACCGAGAAAGACACT | 18 |
| pyc-1 | AATTGTGAGCGGATAACAACAACAGATAGGTGTAAGG | 19 |
| | AGGTTGAGATatgtcacaacagtctattca | |
| pyc-2 | aacgctcggCTTcacttcgat | 20 |
| pyc-3 | atcgaagtgAAGccgagcgtt | 21 |
| pyc-4 | ttattttttcagttcaa | 22 |
| ydeu-U-F | CATAAGCGGGAAGGGTATCGTG | 23 |
| ydeu-U-R | TTGTTATCCGCTCACAATTCCACACATTATACGAGCCGG | 24 |
| | ATGATTAATTGTCAATTGAACCGTGCCGCCATTCTC | |
| ydeu-D-F | TGATGATGAATCATCAGTAAACCGTATAAGCCGCATGTC | 25 |
| | GAGATGGCATGC | |
| ydeu-D-R | ATGTCGTGAGCGTGGTATTGTC | 26 |
| thrA-F | GAATTGTGAGCGGATAACAACGGTAAAGATATCGATAA | 27 |
| | GGAGGTTTTTTatgcgagtgttgaagttcgg | |
| thrB-R | aaacacggGTccacgttgtc | 28 |
| thrB-F | gacaacgtggACccgtgttt | 29 |
| thrC-R | TTACTGATGATTCATCATCA | 30 |
| PGRB-F | Gttttagagctagaaatagcaagttaa | 31 |
| PGRB-R | Attatacctaggactgagc | 32 |
| sgRNA-yeel-1 | AGTCCTAGGTATAATACTAGTAACACAGCAATACGGTACGCGT | 33 |
| | TTTAGAGCTAGAA | |
| sgRNA-yeel-2 | TTCTAGCTCTAAAACGCGTACCGTATTGCTGTGTTACTAGTATTATACC | 34 |
| | TAGGACT | |
| sgRNA-ydeu-1 | AGTCCTAGGTATAATACTAGTTATCTGACCAGTAAATGGGAGTTTTAG | 35 |
| | AGCTAGAA | |
| sgRNA-ydeu-2 | TTCTAGCTCTAAAACTCCCATTTACTGGTCAGATAACTAGTATTATACC | 36 |
| | TAGGACT | |
| sgRNA-yjhE-1 | AGTCCTAGGTATAATACTAGTGCCTATCCGGGCTGTCCCGAGTTTTAG | 37 |
| | AGCTAGAA | |
| sgRNA-yjhE-2 | TTCTAGCTCTAAAACTCGGGACAGCCCGGATAGGCACTAGTATTATA | 38 |
| | CCTAGGACT | |

The present disclosure also provides a culturing method for the above recombinant E. coli, including the following steps: the recombinant E. coli is inoculated on a seed medium and is cultured to obtain a seed liquid; the seed medium includes components with the following concentrations: dried corn steep liquor powder 5 g/L, glucose 20 g/L, yeast powder 5 g/L, KH₂PO₄ 2 g/L, magnesium sulfate 1 g/L, FeSO₄·7H₂O 20 mg/L and MnSO₄·H₂O 20 mg/L.

In some embodiments, an amount of the inoculation in the present disclosure is 20%. In some embodiments, the culturing of the present disclosure is performed at 37° C., and accompanied by shaking. In some embodiments, the shaking is conducted at 500 rpm, and in some embodiments, the culturing is performed for 10 h. After the culturing according to the present disclosure, OD600 is 12-15.

The recombinant E. coli of the present disclosure can use glucose as a substrate to biosynthesize L-threonine, and the production of L-threonine and the glucose conversion rate are significantly improved, laying a foundation for the industrial production of L-threonine.

The present disclosure further provides use of the above recombinant E. coli in the biosynthesis of L-threonine.

E. coli W3110 modified by the present disclosure has high-yielding performance: 1. Overexpression of B. subtilis-derived phosphoenolpck can catalyze phosphoenolpyruvate to generate ketosuccinic acid, and the ketosuccinic acid is threonine acid precursors. Meanwhile, ATP, which is energy, is also generated in the catalytic process of phosphoenolpck, and the ATP is consumed in the process of threonine synthesis, so this technology can not only improve the supply of threonine synthesis precursors, but also provide the ATP energy required in the synthesis process. RBS optimization strategy improves the expression of phosphoenolin E. coli, and point mutation improves thermal stability and catalytic efficiency of the phosphoenol from B. subtilis, such that the conversion of phosphoenolpyruvate to ketosuccinic acid is faster. 2. Overexpression of pyc derived from B. licheniformis can catalyze the synthesis of ketosuccinic acid from pyruvic acid, while there is no pyc in E. coli, so by heterologous expression of pyc, the metabolic pathway for ketosuccinic acid synthesis from pyruvic acid in E. coli is expanded and the accumulation of threonine precursors is increased. The expression of pyc in E. coli is improved by the RBS optimization strategy, and the thermostability and catalytic efficiency of from B. licheniformis are improved by the point mutation, resulting in a faster conversion rate of pyruvic acid to ketosuccinic acid. 3. Overexpression of E. coli endogenous threonine operon thrABA144DC increases the metabolic flux in the direction of threonine synthesis, the expression level of the enzyme is further improved through RBS optimization, and the catalytic efficiency of thrB is improved by point mutation. The above disclosure can improve the ability of the strain to synthesize L-threonine. Meanwhile, these overexpressed genes are integrated into the E. coli genome instead of being overexpressed by plasmids, so there is no need to add antibiotics during the fermentation process to maintain the existence of the plasmids.

The present disclosure further provides a method for biosynthesizing L-threonine, including the following steps: the seed liquid obtained by the above culturing method is inoculated into a fermentation medium, and aerobic fermentation is performed. L-threonine acid is contained in the fermentation liquid.

The fermentation medium includes the components of the following concentrations: glucose 20 g/L, potassium dihydrogen phosphate 2 g/L, yeast powder 3 g/L, betaine 1 g/L, magnesium sulfate 1 g/L, FeSO₄·7H₂O 10 mg/L, MnSO₄·H₂O 10 mg/L, dried corn steep liquor powder 8 g/L and vitamin B1 10 mg/L.

In some embodiments, a volume of the seed liquid of the present disclosure is 20% of that of the fermentation medium, and the aerobic fermentation is performed after the inoculation. The aerobic fermentation is conducted at 37° C. with a dissolved oxygen concentration of 30%. In the process of aerobic fermentation, after substrate sugar is exhausted, residual sugar is controlled at 0˜1 g/L by feeding glucose.

The recombinant nucleic acid of E. coli, the recombinant E. coli and the culturing method thereof, and the method for biosynthesizing L-threonine thereby provided by the present disclosure will be described in detail below in conjunction with examples, but they should not be construed as limiting the claimed scope of the present disclosure.

### Example 1

1. Construction of the Fusion Fragment U-Pck-D

The primers yeeL-U-F, yeeL-U-R, yeeL-D-F and yeeL-D-R in Table 1 were used to amplify the upstream and downstream homology arm fragments on each side of the yeeL gene from the genome of E. coli K-12 W3110 to obtain the fragments yeel1 (SEQ ID NO. 8) and yeel2 (SEQ ID NO. 9), and using the total DNA of E. coli W3110 were used as a template, PCR amplification was conducted with the above primers. The amplification conditions were: pre-denaturation at 95° C. for 5 min; 30 cycles of denaturation at 98° C. for 10 s, annealing at 55° C. for 15 s, extension at 72° C. for 60 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use.

Primers pck-1, pck-2, pck-3 and pck-4 in Table 1 were used to amplify the pck^{G143R} gene from the B. subtilis genome to obtain the fragments 1-pck^{G143R}2-pck^{G143R}, and then with the pck-1 and the pck-4 as primers, and the fragments 1-pck^{G143R} and 2-pck^{G143R} as templates, the fragments 1-pck^{G143R} and 2-pck^{G143R} were fused into pck^{G143R}, where the pck-1 primer contains the optimized RBS sequence, pck-2 and pck-3 were gene point mutation primers of pck, and the RBS optimization and point mutation of the pck^{G143R} gene were conducted by PCR. With the total DNA of B. subtilis as a template, and the above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

Fragments yeel1, pck^{G143R}, yeel2 were subjected to fusion PCR to obtain a fusion fragment U-pck^{G143R}_D (SEQ ID NO: 10), and with yeel1, yeel2, pck^{G143R} as templates, the above primers yeeL-U-F, yeeL-D-R were used for PCR amplification.

The amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use.

2. Construction of Yeel-sgRNA Recombinant Plasmid

Primers pGRB-F and pGRB-R were used to obtain the linearized vector L-pGRB by PCR from the vector pGRB (FIG. 3). The above primers pGRB-F and pGRB-R were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use. The designed sgRNAs (sgRNA-yeel-1 and sgRNA-yeel-2, sgRNA-ydeU-1 and sgRNA-ydeU-2, sgRNA-yjhE-1 and sgRNA-yjhE-2) were ligated with the linearized vector L-pGRB to construct a recombinant plasmid yeel-sgRNA.

3. Construction of Recombinant E. coli LMT2

The recombinant plasmid yeel-sgRNA and fusion fragment U-pck^{G143R}_D were transformed into E. coli K-12 W3110 (LMT1), and primers yeeL-U-F and yeeL-D-R were used to screen the transformants by colony PCR to confirm that the fusion fragment U-pck^{G143R}-D was integrated into the yeel locus successfully. 2 mM arabinose was added for culturing at 30° C. for 12 h, the recombinant plasmid yeel-sgRNA was removed, and the recombinant strain LMT2 was obtained.

Method of plasmid removal: pREDCas9 plasmid was spectinomycin resistant, and pGRB plasmid was ampicillin resistant. 1. The modified strains with both spectinomycin and ampicillin resistances were inoculated in 10 mL of LB medium supplemented with 2 mM arabinose and 1 mM spectinomycin for culturing at 30° C. for 12 h. 2 μl of bacterial liquid was taken to streak on spectinomycin-resistant LB plates for incubation at 30° C. for 12 h, and a single colony was picked to spot-plate on spectinomycin-resistant and ampicillin-resistant LB plates for incubation at 30° C. for 12 h. The colonies that grew normally on the spectinomycin-resistant plate but not on the ampicillin-resistant plate were strains with removed recombinant plasmid pGRB. 2. The strains with removed recombinant plasmid pGRB were inoculated in 10 ml LB medium, and cultured at 42° C. for 12 h. 2 μl of the strain was streaked on an antibiotic-free plate and cultured at 37° C. for 12 h. A single colony was picked to spot-plate on a spectinomycin-resistant plate and the non-antibiotic-resistant plate, for culturing at 37° C. for 12 h, and strains that grew on the antibiotic-free plate but not grew on the spectinomycin-resistant plate were the strains with removed plasmid pREDCas9.

4. Construction of Fusion Fragment U-Pyc-D

Primers yjhE-U-F, yjhE-U-R, yjhE-D-F and yjhE-D-R in Table 1 were used to amplify the upstream and downstream homology arm fragments on each side of the yjhE gene from the genome of E. coli K-12 W3110 to obtain the fragment yjhE 1 (SEQ ID NO: 11) and yjhE 2 (SEQ ID NO: 12). The above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 60 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

Primers pyc-1, pyc-2 and pyc-3, pyc-4 in Table 1 were used to amplify pyc^{A247K} gene from B. licheniformis genome to obtain a fragment pycA247K, the above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

The fragments yjhE 1, pyc^{A247K}, and yjhE 2 were subjected to a fusion PCR to obtain the fusion fragment U-pyc^{A247K}-D (SEQ ID NO: 13). The above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use.

5. Construction of yjhE-sgRNA Recombinant Plasmid

According to the sequence information of the vector PGRB, primers PGRB-F and PGRB-R were designed, and the linearized vector L-PGRB was obtained by PCR from the vector PGRB using the above primers. The designed sgRNA was ligated with the linearized vector L-PGRB to construct a recombinant plasmid yjhE-sgRNA. The above primers were used to PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

6. Construction of Recombinant E. coli LMT3

The recombinant plasmid yjhE-sgRNA and fusion fragment U-pyc^{A247K}-D were transformed into recombinant strain LMT2, and primers yjhE-U-F and yjhE-D-R were used to screen transformants by colony PCR to confirm that the fusion fragment U-pyc-D was integrated into the yjhE locus successfully. 2 mM arabinose was added for culturing at 30° C. for 12 h, then the recombinant plasmid yjhE-sgRNA was removed, and the recombinant strain LMT3 was obtained.

7. Construction of Fusion Fragment U-thrAB^{A144D}C-D

Primers ydeu-U-F, ydeu-U-R, ydeu-D-F and ydeu-D-R in Table 1 were used to amplify the upstream and downstream homology arm fragments on each side of the ydeu gene from the genome of E. coli K-12 W3110 to obtain the fragments ydeu 1 (SEQ ID NO:14) and ydeu 2 (SEQ ID NO. 15). The above primers were used for PCR amplification and the amplification conditions were: pre-denaturation at 95° C. for 5 min; 30 cycles of denaturation at 98° C. for 10 s, annealing at 55° C. for 15 min, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

Primers thrA-F, thrB-R, thrB-F and thrC-R in Table 1 were used to amplify the thrAB^{A144D}C gene from the E. coli genome to obtain the fragment thrAB^{A144D}C. The above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use;

The fragments ydeu 1, thrAB^{A144D}C and ydeu 2 were subjected to a fusion PCR to obtain the fusion fragment U-thrAB^{A144D}C-D (SEQ ID NO: 16). The above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use.

8. Construction of Ydeu-sgRNA Recombinant Plasmid

According to the sequence information of the vector PGRB, primers PGRB-F and PGRB-R were designed. The linearized vector L-PGRB was obtained by PCR from the vector PGRB using the above primers, and the designed sgRNA was ligated with the linearized vector L-PGRB to construct a recombinant plasmid ydeu-sgRNA. The above primers were used for PCR amplification, and the amplification conditions were: pre-denaturation at 95° C. for 5 min; 30 cycles of denaturation at 98° C. for 10 s, annealing at 55° C. for 15 s, extension at 72° C. for 90 s; final extension at 72° C. for 5 min. PCR amplification system: 1 μL of template, 2 μL of upstream and downstream primers, 20 μL of sterilized double distilled water, 25 μL of 2×Phanta Max Master Mix. The PCR product was purified and recovered by gel recovery kit, and the concentration of the recovered product was checked by electrophoresis. The recovered product was stored in a 1.5 mL centrifuge tube and stored in a −20° C. refrigerator for later use.

9. Construction of Recombinant E. coli LMT4

The recombinant plasmid ydeu-sgRNA and the fusion fragment U-thrAB^{A144D}C-D were transformed into E. coli LMT3, and primers ydeu-U-F and ydeu-D-R were used to screen transformants by colony PCR to confirm that the fusion fragment U-thrAB^{A144D}C-D was integrated into the ydeu locus successfully. 2 mM arabinose was added for culturing at 30° C. for 12 h, the recombinant plasmid ydeu-sgRNA was removed, and the recombinant strain LMT4 was obtained.

### Example 2

The recombinant strain LMT4 constructed in Example 1 was inoculated into a seed medium for seed culture, and then the seed culture was transferred to a fermentation medium for culture at an inoculation amount of 20%.

1. Process control of 5 L seed tank
a. A temperature of 37° C., a pH of 7.0, a rotation speed of 500 rpm, and an air volume of 0.3 m3/h were set, the whole process temperature was kept at 37° C., the tank pressure was 0.05˜0.08 MPa, and the culture period was 10 h;
b. Transplantation standard: until OD600 reached 12-15.
c. The seed medium included dried corn steep liquor powder 5 g/L, glucose 20 g/L, yeast powder 5 g/L, KH₂PO₄ 2 g/L, magnesium sulfate 1 g/L, FeSO₄·7H₂O 20 mg/L, and MnSO₄·H₂O 20 mg/L;

2. Fermentation process control of 5 L fermentation tank
a. A temperature of 37° C., a pH of 7.0, an initial speed of 300 rpm, and an air volume of 0.3 m³/h were set, the whole process temperature was kept at 37° C., and the tank pressure was 0.05˜0.08 MPa;
b. control of residual sugar: glucose was added to keep the residual sugar within 0˜1 g/L;
c. control of DO: at 0 h, the air volume was 0.3 m³/h, 300 rpm, and the tank pressure was 0.05 MPa;
d. when the DO dropped below 30%, the dissolved oxygen level was controlled at 30% by adjusting the ventilation rate and stirring speed until the fermentation ended;
e. the fermentation medium included glucose 20 g/L, potassium dihydrogen phosphate 2 g/L, yeast powder 3 g/L, betaine 1 g/L, magnesium sulfate 1 g/L, FeSO₄·7H₂O 10 mg/L, and MnSO₄·H₂O 10 mg/L, dried corn steep liquor powder 8 g/L, vitamin B1 10 mg/L.

3. Determination method of L-threonine:
1) Sample treatment: 1 mL of the fermentation broth after 48 hours of fermentation was centrifuged at 12,000 rpm for 10 minutes to remove bacterial cells and a resulting supernatant was taken. The supernatant was diluted with deionized water and filtered through a filter membrane with a pore size of 0.22 μm.
2) analysis method: pre-derivatization of OPA column
3) chromatographic conditions:
(1) chromatographic column: chromatographic column C18 (250×4.6) mm
(2) column temperature: 40° C.
(3) mobile phase A: 3.01 g of anhydrous sodium acetate was weighed in a beaker, dissolved with ultrapure water and diluted to 1 L, then 200 μL of triethylamine was added, and the pH was adjusted to 7.20±0.05 with 5% acetic acid; after suction filtration, 5 mL of tetrahydrofuran was added, and after mixing, suction filtration was performed again with a 0.22 μm inorganic filter membrane, and then a resulting filtrate was put into an ultrasonic cleaning pot to exhaust for 20 min for later use;

mobile phase B: 3.01 g of anhydrous sodium acetate was weighed in a beaker; dissolved with ultrapure water and diluted to 200 mL; the pH was adjusted to 7.20±0.05 with 5% acetic acid; 400 mL of acetonitrile and 400 mL of methanol was added to this solution for mixing and suction filtration, and a resulting filtrate was put into an ultrasonic cleaning pot to exhaust for 20 minutes for later use.
(4) flow rate: 1.0 ml/min;
(5) UV detector: 338 nm;
(6) column temperature: 40° C.

According to statistics, 160 g/L threonine could be produced in a 5 L fermenter for 48 h, and the glucose conversion rate was 60%.

The above are only the preferred embodiments of the present disclosure. It should be pointed out that for those skilled in the art, several improvements and modifications can be made without departing from the principles of the present disclosure, which should be regarded as the claimed scope of the present disclosure.

What is claimed is:

1. A recombinant nucleic acid of Escherichia coli, comprising a gene encoding phosphoenolpyruvate carboxykinase (pck), a gene encoding pyruvate carboxylase (pyc) and a gene encoding threonine operon.

2. The recombinant nucleic acid according to claim 1, wherein expressions of the gene encoding pck, the gene encoding pyc and the gene encoding threonine operon are all initiated by a Trc promoter.

3. The recombinant nucleic acid according to claim 1, wherein the gene encoding pck is derived from Bacillus subtilis;
the gene encoding pyc is derived from Bacillus licheniformis.

4. The recombinant nucleic acid according to claim 1, wherein the phosphoenolpyruvate carboxykinase pck is RBS optimized and glycine is mutated to arginine at position 143 in the pck;
the pyc is RBS optimized and alanine is mutated to lysine at position 247 in the pyc.

5. The recombinant nucleic acid according to claim 1, wherein the threonine operon is RBS optimized and alanine is mutated to aspartic acid at position 144 in the threonine operon.

6. A recombinant E. coli comprising the recombinant nucleic acid according to any one of claim 1, wherein the recombinant E. coli overexpresses the pck, the pyc and the threonine manipulator.

7. The recombinant E. coli according to claim 6, wherein a basic strain of the recombinant E. coli comprises E. coli K-12 W3110.

8. A culturing method of the recombinant E. coli according to claim 6, comprising the following steps: inoculating the recombinant E. coli on a seed medium for cultivation to obtain a seed liquid; the seed medium comprises components of the following concentrations: dried corn steep liquor 5 g/L, glucose 20 g/L, yeast powder 5 g/L, KH₂PO₄ 2 g/L, magnesium sulfate 1 g/L, FeSO₄·7H₂O 20 mg/L and MnSO₄·H₂O 20 mg/L.

9. A method for biosynthesizing L-threonine, comprising using the recombinant E. coli of claim 6, wherein the recombinant E. coli takes glucose as a fermentation substrate.

10. The method for biosynthesizing L-threonine according to claim 9, comprising the following steps: inoculating the seed liquid obtained by the culturing method according to claim 8 in a fermentation medium, performing an aerobic fermentation to obtain L-threonine, and the L-threonine is in a fermentation liquid;
the fermentation medium comprises components of the following concentrations: glucose 20 g/L, potassium dihydrogen phosphate 2 g/L, yeast powder 3 g/L, betaine 1 g/L, magnesium sulfate 1 g/L, FeSO₄·7H₂O 10 mg/L, MnSO₄·H₂O 10 mg/L, dried corn steep liquor powder 8 g/L and vitamin B1 10 mg/L.

11. The recombinant nucleic acid according to claim 4, wherein the gene encoding pck is derived from B. subtilis.

12. The recombinant E. coli according to claim 6, wherein expressions of the gene encoding pck, the gene encoding pyc and the gene encoding threonine operon are all initiated by a Trc promoter.

13. The recombinant E. coli according to claim 6, wherein the gene encoding pck is derived from B. subtilis.

14. The recombinant E. coli according to claim 6, wherein the pck is RBS optimized and glycine is mutated to arginine at position 143 in the pck;
the pyc is RBS optimized and alanine is mutated to lysine at position 247 in the pyc.

15. The recombinant E. coli according to claim 6, wherein the threonine operon is RBS optimized and alanine is mutated to aspartic acid at position 144 in the threonine operon.

16. The method of claim 8, wherein a basic strain of the recombinant E. coli comprises Escherichia coli K-12 W3110.

17. The method for biosynthesizing L-threonine according to claim 9, wherein a basic strain of the recombinant E. coli comprises E. coli K-12 W3110.
